(19) United States
(12) Patent Application Publication
(10) Pub. No.: US 2024/0140898 A1
(43) Pub. Date: May 2, 2024

(54) METHOD FOR MAKING OMEGA BROMOALKANOIC ACIDS AND ESTERS

(71) Applicant: ARKEMA FRANCE, colombes (FR)

(72) Inventors: Jean-François DEVAUX, Pierre-Benite Cedex (FR); Cyrille LE TOULLEC, Pierre-Benite Cedex (FR); Alain CAMBON, Pierre-Benite Cedex (FR)

(73) Assignee: ARKEMA FRANCE, Colombes (FR)

(21) Appl. No.: 18/004,359

(22) PCT Filed: Jul. 9, 2021
(86) PCT No.: PCT/FR2021/051282

§ 371 Date: Jan. 5, 2023

(30) Foreign Application Priority Data
Jul. 10, 2020 (FR) FR2007324

(51) Int. Cl. C07C 51/363; C07C 51/42; C07C 227/08; C08G 69/08

(52) U.S. Cl. CPC C07C 51/363; C07C 51/42; C07C 227/08; C08G 69/08

(57) ABSTRACT

The invention relates to a process for the continuous synthesis of a compound of formula (II) Br—(CH₂)ₙ₊₂—COOR, comprising a stage consisting of:
(a) the hydrobromination of a compound of formula (I) CH₂═CH—(CH₂)ₙ—COOR:
   where, in the formulae (I) and (II), n is an integer of between 7 and 9, and R is chosen from H or a linear or branched alkyl radical comprising from 1 to 10 carbon atoms, in particular methyl, ethyl, isopropyl or propyl,
   with HBr in the presence of a radical initiator and of at least one solvent;
      said process being characterized in that the reaction is carried out in the absence of benzene and of toluene and in that, in stage (a), the HBr is injected into the reaction mixture in the gaseous form and in stoichiometric excess.

### TECHNICAL FIELD

The present patent application relates to a process for the continuous production of ω-bromoalkanoic acids and esters by hydrobromination. It also relates to a process for the production of aminocarboxylic acids and esters and of polyamide or of copolyamide from said ω-bromoalkanoic acids or esters.

### PRIOR ART

ω-Bromoalkanoic acids or esters, in particular the compounds of formula (II) below:

Br—(CH₂)ₙ+₂—COOR (II)

are advantageous precursors in the polymer industry. In particular, they constitute intermediates for the amino acids and amino esters necessary for the manufacture of polyamides. Thus, 11-bromoundecanoic acid is the precursor of 11-aminoundecanoic acid, used on the industrial scale for the manufacture of polyamide 11.

These compounds can be obtained by hydrobromination of a terminally unsaturated carboxylic acid or ester, of formula (I):

CH₂═CH—(CH₂)ₙ—COOR (I)

in which n is an integer of between 7 and 9, R is chosen from H or a linear or branched alkyl radical comprising from 1 to 10 carbon atoms. The hydrobromination is carried out by addition of anti-Markovnikov type of HBr to the compound of formula (I) in the presence of a radical initiator and of one or more solvents.

Although it is possible to carry out the hydrobromination in batch mode, this requires recurrent interventions, poses difficulties in recovering the residual gaseous HBr and is problematic to control because of the high exothermicity of the reaction. The hydrobromination is thus generally carried out continuously.

The already old patent FR 928 265 describes the hydrobromination of 10-undecenoic acid continuously in a column maintained at a temperature of 30° C. through which a solution of 10-undecenoic acid in toluene passes and, countercurrentwise, HBr in excess and air. This process works well but produces approximately 20% of 10-bromoundecanoic acid, which severely limits the yield.

Semyonov et al. provide (Maslozhirova Promyshlennost, 1971, Vol. 37, pp. 31-33) such a process having a markedly higher yield (>90%) in which the reaction is carried out in toluene in a plug-flow reactor at a temperature of 0-5° C. Cooling the reactor to a very low temperature makes this process not very energy efficient and requires major capital expenditures.

The patent CN 103804209 B describes the hydrobromination of 10-undecenoic acid continuously in a system of two stirred reactors in series. The injection is carried out of a mixture of 10-undecenoic acid in toluene and benzene, 1% to 5% by weight of azobisisobutyronitrile or benzoyl peroxide as radical initiator and of HBr into the first stirred reactor, maintained at a temperature of 10-30° C., with a residence time of 30 to 90 minutes. The reaction medium from the first reactor is withdrawn continuously and injected into an item of separation equipment heated to 65-80° C. The residual HBr released in the gas form is returned to the first reactor. The maximum yield indicated is 92.1%. This process requires a prolonged residence time for a moderate yield. Furthermore, the large amount of radical initiator can be the source of troublesome residues in the product.

All of these continuous processes operate with benzene, a carcinogenic and mutagenic solvent, and/or toluene, a solvent capable of producing benzyl bromide, a lachrymatory compound.

In point of fact, today the aim is increasingly to replace benzene and toluene with other solvents having a more favorable toxicity profile or generating less in the way of byproducts.

The patent EP 3 030 543 B1 provides a process for the continuous hydrobromination of 10-undecenoic acid which makes it possible to at least partially replace benzene with cyclohexane and/or methylcyclohexane. In this process, 10-undecenoic acid is reacted with HBr in liquid form. The document teaches that the implementation of the process in a countercurrent column with modification of solvent leads to a loss in yield, which can be compensated for when two successive reactors are used, the first with a turbulent flow and the second a laminar flow. This method exhibits the disadvantage of requiring an HBr in liquid form, which has the consequence of constraints in terms of HBr purity and considerable energy expenditures and capital costs in order to cool the HBr or an HBr solution to temperatures markedly below 0° C. in order for the solubility of the HBr to be sufficient.

### SUMMARY OF THE INVENTION

An aim of the invention is thus to provide a process for the continuous synthesis of ω-bromoalkanoic acids and esters by hydrobromination not employing benzene and/or toluene, which exhibits a satisfactory yield of product of formula (II), preferably of at least 92% and in particular of at least 94%.

According to one embodiment, an aim of the invention is to provide a continuous synthesis process which saves energy, in particular requiring neither high pressure nor a temperature below 5° C.

According to another embodiment, an aim of the invention is to provide a continuous synthesis process making possible the use of HBr contaminated with hydrogen, HCl or water.

According to another embodiment, an aim of the invention is to provide a continuous synthesis process making it possible to reduce the amount of HBr introduced into the process, this compound being expensive to produce and to remove.

According to yet another embodiment, an aim of the invention is to provide a continuous synthesis process with a reduced residence time, in particular of less than 30 minutes and very particularly of less than 15 minutes.

According to another embodiment, an aim of the invention is to provide a continuous synthesis process making it possible to produce a compound of formula (II) comprising nothing or little in the way of impurities.

According to another embodiment, an aim of the invention is to provide a continuous synthesis process not requiring solid radical initiators, which are reagents at risk of violent decomposition.

According to another embodiment, an aim of the invention is a process for the production of aminocarboxylic acid or ester from a compound of formula (II).

Finally, according to another embodiment, an aim of the invention is a process for the production of a polyamide or copolyamide from a compound of formula (II).

In point of fact, the present invention is based on the observation that it is possible to replace benzene and toluene, in the continuous production of ω-bromoalkanoic acids and esters by hydrobromination, with aliphatic solvents while maintaining a high yield under the condition of ensuring a sufficient molar excess of HBr during the reaction.

In order to obtain a high yield of compound of formula (II), it is important to properly control the thermal conditions because a high temperature promotes the appearance of entities not carrying bromine at the chain end.

Consequently, according to a first aspect, a subject matter of the invention is a process for the continuous synthesis of a compound of formula (II) Br—(CH₂)ₙ+₂—COOR, comprising a stage consisting of:
(a) the hydrobromination of a compound of formula (I) CH₂═CH—(CH₂)ₙ—COOR:
where, in the formulae (I) and (II), n is an integer of between 7 and 9, and R is chosen from H or a linear or branched alkyl radical comprising from 1 to 10 carbon atoms, in particular methyl, ethyl, isopropyl or propyl,
with HBr in the presence of a radical initiator and of at least one solvent;
   said process being characterized in that the reaction is carried out in the absence of benzene and of toluene and in that, in stage (a), the HBr is injected into the reaction mixture in the gaseous form and in stoichiometric excess.

Advantageously, the ratio of the molar flow rate of HBr injected in stage (a) to the molar flow rate of the compound of formula (I) injected in stage (a) is from 1.2 to 3, preferably from 1.3 to 2.2, more preferentially from 1.4 to 2 and in particular from 1.5 to 1.9.

According to one embodiment, the outlet stream from the reactor of the liquid reaction mixture on conclusion of stage (a) comprises at least 2%, preferably at least 3%, and more preferentially at least 3.5% and in particular at least 4% by weight of HBr.

Preferably, the process of the invention additionally comprises the subsequent stages consisting of:
(b) the separation of the excess HBr from the liquid reaction mixture resulting from stage (a);
(b1) optionally, separation of the excess HBr from the gaseous reaction mixture resulting from stage (a); and
(c) the recycling of the HBr separated in stage (b) and (b1) if appropriate to stage (a).

Advantageously, the process of the invention comprises the stages consisting of:
(a1) introduction of the compound of formula (I), of the HBr, of the initiator and of the solvent(s) into a first reactor at an appropriate temperature for an appropriate residence time;
(a2) withdrawal of the reaction mixture from the first reactor and introduction into an item of separation equipment; if appropriate, followed by a subsequent stage of:
(b) separation of the residual HBr from the reaction mixture; and
(c) recycling of the separated HBr to stage (a1).

Stage (a) can be carried out in a reaction medium saturated with HBr. It can be carried out at a temperature of between 5 and 50° C., preferably between 10 and 40° C. and very particularly from 20 to 30° C.

The radical initiator can be molecular oxygen used as is or as a mixture with inert gases, for example air or oxygen-enriched air.

The first reactor can in particular be a stirred vessel with a self-priming turbine or a jet loop reactor comprising a venturi. The item of separation equipment can in particular be a stirred vessel or a column.

Advantageously, the process according to the invention is carried out in the absence of aromatic solvent.

The product of formula (I) can be chosen from 11-bromoundecanoic acid, 10-bromodecanoic acid and 9-bromononanoic acid.

The solvent can be chosen from cyclohexane, methylcyclohexane, methylcyclopentane, n-hexane, 2-methylhexane, 3-methylhexane, n-heptane, isooctane, petroleum ether, tetralin, 1,1,1-trichloroethane, dibromoethane, chloroform, carbon tetrachloride, tetrachlorethylene, 1-bromopropane, dimethyl carbonate, tetrahydrofuran, 1,4 dioxane, 2-methyltetrahydrofuran, tetrahydropyran, 1-propoxypropane, 1-ethoxybutane, 2-isopropoxypropane, acetonitrile and their mixtures.

According to another aspect, the invention relates to a process for the synthesis of a compound of formula (III) NH₂—(CH₂)ₙ+₂—COOR, comprising a stage consisting of:
(i) ammonolysis on the compound of formula (II) obtained by the above process; and
(ii) separation of the compound of formula (III) NH₂—(CH₂)ₙ+₂—COOR formed.

Finally, according to another aspect, the invention relates to a process for the synthesis of a polyamide or copolyamide comprising the stage of polycondensation of the compound of formula (III) obtained by the above process, alone or as a mixture with other monomers.

### BRIEF DESCRIPTION OF THE FIGURES

A better understanding of the invention will be obtained in the light of the description which follows and of the figures, which show:

FIG. 1: a diagram of an installation for the implementation of a process according to one embodiment of the invention;

FIG. 2: a diagram of an installation for the implementation of a process according to one embodiment of the invention comprising a venturi and an external heat exchanger;

FIG. 3: a diagram of an installation for the implementation of a process according to one embodiment of the invention comprising a venturi.

### DESCRIPTION OF THE EMBODIMENTS

### Definition of the Terms

In the context of the present disclosure, the term “stoichiometric excess, in the context of a continuous process,” is understood to mean a greater molar flow rate of reactant than that required for the reaction envisaged. For example, one mole/hour of HBr is required to carry out the hydrobromination of one mole/hour of compound of formula (I). Consequently, a ratio of the molar flow rate of HBr/molar flow rate of the compound of formula (I)>1 constitutes a stoichiometric excess of HBr.

In the context of the present disclosure, the term “residence time” is understood to mean the ratio of the volume occupied by the liquid reaction mixture to the sum of the flow rates by volume of compound of formula (I) and of solvents introduced into the process.

In the context of the present disclosure, the term “ω-bromoalkanoic acids or esters” is understood to denote alkanoic acids or esters carrying at least one bromine atom on the terminal carbon atom. ω-Alkanoic acids or esters having a linear chain are preferred.

The process of the invention is targeted at producing in particular ω-bromoalkanoic acids or esters of formula (II) below:

Br—(CH₂)ₙ+₂—COOR (II)

in which n is an integer of between 7 and 9, and R is chosen from H or a linear or branched alkyl radical comprising from 1 to 10 carbon atoms, in particular methyl, ethyl, isopropyl or propyl.

The process is particularly advantageous for the manufacture of 12-bromododecanoic acid, 11-bromoundecanoic acid and 10-bromodecanoic acid.

Compound of Formula (I)

ω-Bromoalkanoic acids or esters of formula (II) can be obtained by hydrobromination of a terminally unsaturated carboxylic acid or ester, of formula (I):

CH₂═CH—(CH₂)ₙ—COOR (I)

in which n is an integer of between 7 and 9, and R is chosen from H or a linear or branched alkyl radical comprising from 1 to 10 carbon atoms, in particular methyl, ethyl, isopropyl or propyl.

The compound of formula (I) is advantageously 10-decenoic acid, 11-undecenoic acid or 12-dodecenoic acid or one of their esters, in particular their methyl, ethyl, isopropyl or propyl ester.

These compounds are commercially available or can be synthesized by employing conventional organic chemistry reactions. Some of these compounds can be obtained from starting materials which are sustainable because of plant origin. Thus, 11-undecenoic acid is advantageously from castor oil, as described in particular in FR 952 985.

The compounds of formula (I) are preferably employed in liquid form, either in molten form or in solution in a suitable solvent.

Advantageously, the compound of formula (I) is employed at a temperature of 10 to 70° C. and in particular of 20 to 50° C.

HBr

The HBr is commercially available or can be produced by reaction of bromine with hydrogen or be the coproduct of another reaction, for example the bromination of an aromatic compound. When the process of the invention is used for the manufacture of aminocarboxylic acids from ω-bromoalkanoic acid, in particular for the manufacture of polyamides, the HBr can be obtained advantageously by:
(i) reaction of the ω-bromoalkanoic acid with ammonia in aqueous solution to form a reaction mixture comprising the corresponding ω-aminocarboxylic acid and ammonium bromide;
(ii) separation from the reaction mixture of the ω-aminocarboxylic acid and of an aqueous solution rich in ammonium bromide;
(iii) bringing the aqueous solution rich in ammonium bromide obtained into contact with sodium hydroxide to form ammonia and an aqueous solution rich in sodium bromide;
(iv) purification of the aqueous solution rich in sodium bromide obtained in order to remove the organic impurities;
(v) bringing the purified aqueous solution rich in sodium bromide obtained into contact with chlorine to form bromine and an aqueous solution rich in sodium chloride; and
(vi) reaction of the bromine obtained with hydrogen to form hydrogen bromide.

The HBr can be used pure but one of the advantages of the process of the invention is that it also makes possible its use as a mixture with other gases, such as hydrogen, HCl, carbon dioxide or water. Generally, the total content of the HBr in other gases is nevertheless less than 30 mol %, advantageously less than 20 mol % and very particularly less than 10 mol %, with respect to the HBr. Furthermore, the water content of the HBr is advantageously less than 3 mol %, advantageously 1 mol %, with respect to the HBr.

According to the invention, the HBr is introduced into the reaction mixture in stage (a) in gaseous form. Nevertheless, the HBr can dissolve partially or completely in the reaction medium comprising the compound of formula (I), the solvent and also the product of formula (II), which reaction medium is generally in liquid form.

The flow rate of HBr injected into the reaction mixture in stage (a) is the sum of the HBr introduced into the process and, if appropriate, of the recycled HBr.

The inventors have found that the selectivity and for this reason the yield of the hydrobromination reaction is promoted in the presence of a high amount of HBr dissolved in the reaction medium, which is obtained by injecting a large stoichiometric excess of HBr into the reaction medium.

According to the process of the invention, the ratio of the molar flow rates of the HBr injected into the reaction mixture in stage (a) to the compound of formula (I) injected in stage (a) will generally be from 1.2 to 3, preferably from 1.3 to 2.2, more preferentially from 1.4 to 2 and in particular from 1.5 to 1.9. Here and in what follows, the molar ratio of pure HBr with the compound of formula (I), with the exclusion of any other gas or humidity possibly present, is meant.

Once injected into the reaction mixture, the HBr can dissolve in the reaction mixture and be available for the targeted reaction. The HBr in excess of its solubility in the reaction mixture or which does not manage to dissolve in the reaction medium can be evacuated from the reactor in the gas form, in particular in order to regulate the pressure.

Furthermore, the part of the other gases introduced with the HBr which has not dissolved in the reaction medium can likewise be evacuated from the reactor.

Advantageously, the HBr not consumed by the reaction remains in the reaction mixture as residual HBr and can then be evacuated in this form. Advantageously, the outlet stream from the reactor of the liquid reaction mixture comprises at least 2%, preferably at least 3%, and more preferentially at least 3.5% and in particular at least 4% by weight of HBr, with respect to the weight of the liquid outlet stream from the reactor.

This is because it has been found that, under these conditions, the yield of compound of formula (II) was at a maximum.

As will be explained in greater detail below, it is possible to recycle the residual HBr, after separation from the liquid reaction mixture withdrawn from the reactor. Similarly, it is possible to recycle a portion of the HBr evacuated from the reactor in gas form.

The amount of recycled HBr can vary, depending on the molar ratio of total HBr injected into the reaction medium, on the gas/liquid transfer, but also, if appropriate, on the conditions of separation of the reaction mixture. Preferably, the recycled HBr exhibits a molar ratio with the compound of formula (I) of greater than 0.2 and less than 1.5. Generally, this molar ratio will be from 0.3 to 1, preferably from 0.4 to 0.9, more preferentially from 0.5 to 0.8.

When the process of the invention is carried out with recycling of the HBr, the HBr introduced into the process is preferably injected in stoichiometric excess and thus exhibits a molar ratio with the compound of formula (I) of greater than 1. Generally, this molar ratio will be from 1.01 to 1.5, preferably from 1.02 to 1.4, more preferentially from 1.03 to 1.3 and in particular from 1.03 to 1.2.

The inventors have discovered that the use of the recycling of HBr makes it possible to increase the selectivity and the yield while minimizing the consumption of HBr and the discharges to the environment of the excess HBr.

Several means make it possible to adjust the flow rate of residual HBr in the liquid outlet stream from the reactor.

The flow rate can be determined by the usual means of analysis of HBr, in particular by argentometry, acid-base titration or ion chromatography.

When the content of residual HBr in the liquid outlet stream from the reactor is judged to be too low, the flow rate of HBr introduced into the process can be increased.

Alternatively, if the liquid phase containing the compound (II) after the stage of separation of the HBr described below contains more than 0.1% by weight of HBr, the flow rate of recycled HBr can be increased by improving the conditions of separation of the residual HBr during the stage of separation of the HBr, for example by increasing the temperature of the stage of separation of the HBr, as indicated below.

In a specific embodiment of the invention, a stoichiometric excess of HBr in the reaction medium of stage (a) is ensured by monitoring the flow rate of HBr evacuated from the reactor in gas form. This monitoring can be carried out, for example, by measuring the total gas flow evacuated from the reactor and the HBr concentration of this gas. The ratio of the gaseous molar flow rate of HBr evacuated from the reactor to that of the HBr introduced into the reactor is preferably of between 0.01 and 0.5, more preferentially of between 0.02 and 0.4, more preferentially of between 0.03 and 0.3 and in particular of between 0.03 and 0.2.

Solvents

According to the invention, the process does not employ benzene or toluene.

In general, the appropriate solvents for the process of the invention are inert organic solvents which dissolve the compound of formula (I) and the product of the reaction of formula (II) and also the HBr at the temperature of the reaction.

Appropriate solvents can be chosen from aliphatic or cycloaliphatic compounds, in particular linear or branched alkanes comprising from 1 to 10 carbon atoms, if appropriate substituted by one or more halogen atoms, in particular bromine or chlorine atoms, alkoxy groups or nitrile groups; cycloaliphatic compounds, in particular cycloalkanes comprising a ring of 4 to 8 carbon atoms which is optionally substituted and/or interrupted, in particular by one or more oxygen atoms. Some solvents can be esters, in particular carbonate esters.

Mention may in particular be made, among the suitable solvents, of cyclohexane, methylcyclohexane, methylcyclopentane, n-hexane, 2-methylhexane, 3-methylhexane, n-heptane, isooctane, petroleum ether, tetralin, 1,1,1-trichloroethane, dibromoethane, chloroform, carbon tetrachloride, tetrachloroethylene, 1-bromopropane, dimethyl carbonate, tetrahydrofuran, 1,4-dioxane, 2-methyltetrahydrofuran, tetrahydropyran, 1-propoxypropane, 1-ethoxybutane, 2-isopropoxypropane, acetonitrile, fluorobenzene, chlorobenzene, trifluorotoluene, ethylbenzene, o-xylene, m-xylene, p-xylene and their mixtures.

In the process of the invention, benzene is not used, insofar as it is a carcinogenic and mutagenic product, nor is toluene, insofar as the latter can form benzyl bromide, which is strongly lachrymatory and is difficult to separate from the reaction product and the solvent.

Advantageously, the process does not employ solvents presenting HSE problems and/or aromatic solvents.

Preferably, the solvent used is chosen from cyclohexane, methylcyclohexane, methylcyclopentane, 2-methylhexane, 3-methylhexane, n-heptane, isooctane, petroleum ether and their mixtures. Cyclohexane and methylcyclohexane are particularly preferred.

The ratio of the flow rates by weight of the compound of formula (I) to the solvent participating in the process can vary widely and it can be determined according to the conditions of the process by routine tests. In principle, a ratio of the flow rates by weight of the compound of formula (I) to the solvent participating in the process of from 1:1 to 1:20 and preferably from 1:2 to 1:10 and very particularly from 1:3 to 1:6 is suitable. It will generally be preferred to work under concentrated conditions in order to optimize productivity. Nevertheless, it is preferable for the amount of solvent to be sufficient so as to prevent the compound of formula (I) or (II) from crystallizing in particular in the reaction stage.

The solvent participating in the process can be injected into the reaction medium separately or else as a mixture with the compound (I).

Advantageously, the solvent injected into the reaction medium contains recycled HBr as described below.

Radical Initiator

The hydrobromination reaction generally requires the presence of a radical initiator.

The radical initiator can be chosen, for example, from oxygen, an oxygen-containing gas, such as air, a peroxide, such as benzoyl peroxide, a diazo compound, such as azobisisobutyronitrile, or any other radical generator, such as UV rays.

Molecular oxygen or an oxygen-containing gas, such as air or oxygen-depleted air, constitute preferred radical initiators because they are readily available, inexpensive and produce little or nothing in the way of residues in the product, and do not present a problem of stability on storage.

The amount of radical initiator is that conventionally used.

When it is a peroxide or a diazo compound, it can be employed in an amount of between 0.1% and 4% by weight, with respect to the weight of the compound of formula (I).

When oxygen or an oxygen-containing gas is used as radical initiator, its amount, expressed as molar ratio of oxygen with the amount of HBr introduced into the process (thus excluding optionally recycled HBr), can vary in particular between 1:5000 and 1:50 and preferably between 1:1000 and 1:200.

Items of Equipment and Process Conditions

Stage (a) of hydrobromination of the process of the invention can be carried out very simply by continuously bringing the compound of formula (I) into contact with HBr in the presence of the radical initiator and of one or more solvents.

Given the gaseous form of the HBr, the stage is advantageously carried out in a reactor promoting gas/liquid material transfer. Such reactors can, for example, be based on a column, such as a spray column, a falling film column, a bubble column, an ejector column, a mechanically stirred column, a countercurrentwise or cocurrentwise packed column or also a perforated-plate column.

Alternatively, it can also be a reactor based on a stirred vessel, for example equipped with a turbine mixer or with a venturi ejector.

Finally, it can be a loop reactor, if appropriate fitted with an ejection nozzle or with a venturi ejector (jet loop reactor). This type of reactor comprises a vessel in which a pump continuously withdraws the liquid reaction medium optionally comprising a gaseous fraction in the form of bubbles in order to return it to an ejector connected to the gaseous stream of HBr injected into the reaction medium. In the ejector, the liquid reaction mixture is ejected at high speed and the gaseous stream of HBr is dispersed in the reaction medium in the form of fine bubbles. The output from the ejector is sent to the vessel. Preferably, a pipe connects the gas phase of the vessel with the gas inlet of the ejector.

Preferably, the hydrobromination stage is carried out in a vessel stirred with a turbine or in a jet loop reactor, in particular a jet loop reactor with a venturi ejector.

Temperature

The temperature of the reactor during the hydrobromination stage is preferably set above the crystallization temperature of the reactant and of the products. Furthermore, it is preferred to choose a temperature which is not excessively low in order to limit energy expenditures. It is preferred to choose a temperature which is not excessively high so as to ensure good selectivity. Generally, the temperature during the reaction stage will preferably be from 5 to 50° C., preferably from 10 to 40° C. and in particular from 20 to 30° C.

The hydrobromination reaction of the compound of formula (I) is highly exothermic. In order to ensure good selectivity, it is advantageous to couple the reactor to a heat-exchange device. Such devices are known to a person skilled in the art; it can, for example, be a jacket around the reactor, or a device located on an exterior loop, or also inside the reactor. Preferably, the heat-exchange device is located on an exterior loop, outside the reactor. In this case, liquid reaction medium is withdrawn continuously from the reactor, sent to the external exchanger and then returned to the reactor. Any type of heat exchanger can be envisaged, such as, for example, tube-type or plate exchangers.

According to a specific embodiment of the invention, it will be possible to use a packed column into which the solvent and the reactant (I) are injected at the top and, at the bottom, the HBr and the radical initiator and in which a portion of the liquid reaction medium is withdrawn at the bottom of the column and sent, using a pump, to a heat exchanger and reinjected at the top of the column.

According to another specific embodiment of the invention, the reactor used will be a jet loop reactor comprising a heat exchanger between the pump and the ejector.

Advantageously, the compound of formula (I) is added in liquid form. A compound of formula (I) with a melting point at 10° C. or less can be added at a temperature close to ambient temperature, that is to say from 15 to 35° C. A compound of formula (I) with a melting point of greater than 10° C. is preferably heated before introduction into the reactor, for example to a temperature 25° C. above its melting point. The solvent is preferably introduced into the reactor with a temperature of 5 to 35° C. and preferably close to ambient temperature, that is to say from 15 to 35° C.

Pressure

The pressure in the reactor during the hydrobromination stage (a) is generally of between 0.5 and 5, preferably between 0.9 and 3 and in particular from 1 to 1.5 bar absolute. Advantageously, the reactor is at an absolute pressure between 1.05 and 1.25 bar absolute.

Advantageously, the installation provided for the implementation of the process comprises at least one gas vent, in order to control the pressure. It is thus possible to keep the pressure constant by removing the excess gas, in particular the unreactive gaseous compounds introduced by the HBr, and also the HBr which has not dissolved in the reaction medium.

Residence Time

Advantageously, the hydrobromination stage (a) of the process of the invention makes it possible to achieve an almost complete conversion of the compound of formula (I) with a reduced residence time.

Thus, the residence time in the reactor is generally of between 1 and 60 minutes and preferably between 2 and 45 minutes and preferably between 5 and 30 minutes.

HBr Recycling

It has been discovered that the hydrobromination process could be carried out with different solvents from toluene and benzene without significant loss of yield if a sufficient content of HBr dissolved in the reaction medium at the outlet of the reactor was ensured. Under these conditions, it has been observed that it is possible to obtain a high selectivity and thus a high yield of product of formula (II).

While it is of course possible to increase the amount of HBr injected into the reactor until the desired yield is obtained, this generates costs associated with the production of the HBr and its removal from the reaction mixture.

Consequently, according to a specific embodiment of the process of the invention, provision is made to recycle the residual HBr in the reaction mixture withdrawn from the reactor in order to increase the molar ratio of HBr injected into the reactor of stage (a) without an associated additional cost. The accumulation of recycled residual HBr associated with the use of HBr in stoichiometric excess can result in the concentration of HBr in the reaction medium of stage (a) being close to the solubility of HBr in the reaction medium at the temperature and the pressure under consideration.

Stage (b) of separation of the residual HBr from the reaction mixture can be carried out in a suitable item of separation equipment, for example a stirred vessel, an exchanger and flash drum device, or also, preferably, a column equipped with a packing or with plates and comprising a reboiler at the bottom.

The recycling of the HBr can be carried out by withdrawing the reaction mixture from the hydrobromination reactor and sending it to the item of separation equipment, in which the HBr can be separated from the reaction mixture by simple heating. Preferably, the liquid mixture is heated to a temperature close to the boiling point of the solvent, so as to evaporate more than 70%, preferably more than 80% and preferably more than 90% and in particular more than 99% of the residual HBr present in the reaction stream. The gaseous HBr thus recovered can subsequently be returned to the first reactor by conventional means. It would remain within the scope of this invention if the gaseous stream resulting from the separation stage were cooled, thus inducing an at least partial condensation of solvent entrained in this gaseous stream, the gaseous and liquid streams being returned to stage (a).

At the end of this stage of separation of the residual HBr, a liquid stream of product of formula (II) and of solvent is furthermore recovered. This liquid stream can be subjected to a washing stage followed by a stage of separation by settling, for example with water or a dilute aqueous sodium hydroxide solution, in order to remove the traces of residual HBr. The solvent can be removed, for example by evaporation, and then, if appropriate, be recycled in the reaction. The crude product of formula (II) recovered can subsequently be purified by conventional means, in particular by crystallization in the molten state or by recrystallization, in particular from the reaction solvent, or else used as is without a purification stage.

According to another specific embodiment of the process of the invention, provision is made to at least partially recycle the HBr present in the gaseous stream exiting from stage (a) by at least partially separating the HBr contained in the gaseous stream resulting from the reaction mixture of stage (a) and by returning the separated HBr to stage (a). Said gaseous stream exiting from stage (a) is advantageously brought into contact with optionally recycled solvent in order to absorb a portion of the HBr. This operation of bringing into contact can be carried out by means known to a person skilled in the art, such as, for example, a packed column. The stream of solvent enriched in HBr thus obtained can subsequently be sent to stage (a). Thus, the stoichiometric excess of HBr introduced into the reaction and the efficiency of the separation of the HBr in the item of separation equipment make it possible to control the excess of HBr dissolved in the reaction medium in order to optimize the yield of product of formula (II).

The product of formula (II) can undergo an ammonolysis reaction by reaction with ammonia to form the corresponding ω-aminocarboxylic acid or ester of formula (III). The compound of formula (III), after having optionally undergone purification stages, can be polymerized, for example by polycondensation, to give the corresponding polyamide. Alternatively, it can also be employed with other monomers, such as, for example, diamines and dicarboxylic acids, one or more lactams or polyethers for the manufacture of corresponding copolymers.

The process according to the invention makes it possible to obtain a product of formula (II) comprising fewer impurities, which simplifies the purification stages before the reaction with ammonia or, if it is used without purification, after the reaction with ammonia.

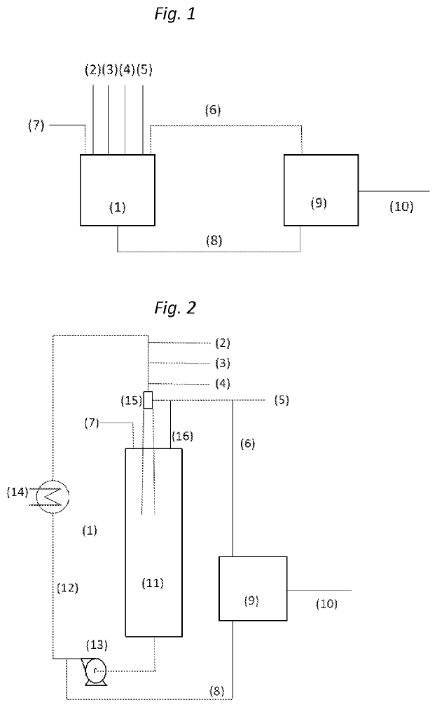

In the embodiment of the continuous process of the invention illustrated in FIG. 1, a hydrobromination reactor (1) comprises a continuous feed (2) of compound of formula (I), a continuous feed (3) of solvent, a continuous feed (4) of initiator, a continuous feed (5) of gaseous HBr introduced into the process and a continuous feed (6) of recycled HBr in gaseous form. The reactor furthermore comprises a gas vent (7) making it possible to remove the excess gas arriving at the reactor. It comprises a liquid withdrawal (8) of reaction mixture sent to an item of equipment (9) for separation of HBr which comprises a withdrawal of HBr (6) and a withdrawal (10) of liquid phase containing the compound of formula (II) and the solvent.

In the embodiment of the continuous process of the invention illustrated in FIG. 2, a hydrobromination reactor (1) comprises a vessel (11) provided with a recirculation loop (12) with a pump (13), the suction of which is connected to the vessel (11) and the discharge of which to a heat exchanger (14), which is connected to a venturi (15) joined onto the reactor. A continuous feed (5) of HBr introduced into the process and a continuous feed (6) of recycled HBr and a loop for equilibration (16) of the gaseous headspace of the reactor are connected to the gas suction of the venturi. The continuous feeds (2) of solvent, (3) of compound of formula (I) and (4) of initiator are connected to the line between the heat exchanger and the venturi. A line (8) for liquid withdrawal of the reaction mixture is connected to a vessel (9) for separation of HBr which comprises a continuous withdrawal (6) of recycled HBr in gaseous form and a continuous withdrawal (10) of liquid phase containing the compound of formula (II) and the solvent.

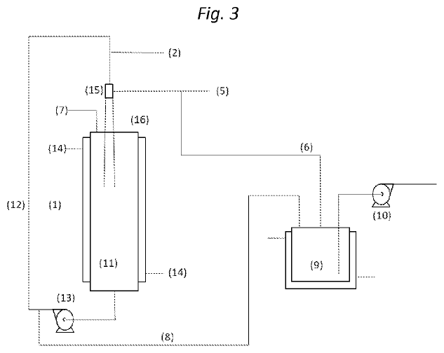

In the example of implementation of the continuous process of the invention illustrated in FIG. 3, a hydrobromination reactor (1) comprises a jacketed vessel (11) cooled by a continuous feed (14) of heat-exchange fluid, a recirculation loop (12) with a pump (13), the suction of which is connected to the vessel (11) and the discharge of which to a venturi (15) joined onto the reactor. A continuous feed (5) of gaseous HBr introduced into the process and (6) of recycled HBr in gaseous form and a loop for equilibration (16) of the gaseous headspace of the reactor are connected to the gas suction of the venturi. The continuous feed (2) of a mixture of compound of formula (I), solvent and initiator is connected to the line between the heat exchanger and the venturi. A line (8) for liquid withdrawal of reaction mixture is connected to a second jacketed vessel (9) for separation of HBr which comprises a continuous withdrawal of HBr (6) in gaseous form and a continuous withdrawal of liquid phase containing the compound of formula (II) and the solvent by a pump (10).

The invention will be explained in greater detail in the examples which follow.

### EXAMPLES

### Example 1

A hydrobromination of 10-undecenoic acid is carried out in an installation as illustrated in FIG. 3, explained below. The venturi (15) is a glass filter pump (waterjet pump reference 181-9205 from VWR International), the liquid outlet of which is connected to the cylindrical jacketed vessel (11). The recirculation pump (13) has a flow rate of 100 I/h.

A flow rate of 2361 g/h of a 15% by weight solution of 10-undecenoic acid in cyclohexane at ambient temperature and a stream of air are injected via the feed (2). The gaseous HBr introduced into the process is continuously injected at a flow rate such that the ratio of the flow rate of gaseous HBr in mol/h to the flow rate of 10-undecenoic acid in mol/h is 1.15. The ratio of the flow rate by volume of HBr to the flow rate by volume of air is 35:1.

The vessel (11) is maintained at a pressure 0.1 bar above atmospheric pressure by the vent (7) on the gas phase of the reactor connected to the atmosphere by a vent processing system. Throughout the duration of the experiment, the temperature in the vessel (11) is kept constant at 24° C. by virtue of the circulation in the jacket of a heat-exchange fluid. The volume of reaction medium in the vessel (11) and of the loop (12) is kept constant at 0.3 liter by continuous export (8) to a separation vessel (9). The residence time in the first reactor is of the order of 6 minutes.

The separation vessel (9) is stirred by a magnetic bar and heated by the circulation of heat-exchange fluid in the jacket so as to keep the temperature of the reaction liquid at 80° C. The liquid level in the vessel (9) is kept at 0.15 liter by continuous export of the reaction mixture using the pump (10). At start-up, the vessel (11) and the loop contain cyclohexane saturated with HBr, and the vessel (9) is empty.

In order to determine the concentration of residual HBr and the performance qualities of the hydrobromination reaction at the outlet of the reactor, after 60 minutes an aliquot of liquid reaction mixture is withdrawn on the line (8) and subjected to analysis by argentometry and by gas chromatography.

Argentometric analysis makes it possible to determine the concentration by weight of HBr in the aliquot. It is carried out by diluting the aliquot to 30% in demineralized water, by shaking vigorously, then, after separation by settling, by withdrawing half of the aqueous phase and by diluting it by a factor of 10 in demineralized water and titrating with a 0.1N aqueous silver nitrate solution.

Analysis by gas chromatography is carried out by derivatization of 0.1 ml of liquid reaction mixture by 1 ml of N,O-bis(trimethylsilyl)trifluoroacetamide with 1% of trimethylchlorosilane for 30 minutes at 80° C., then injection on a nonpolar column and detection by flame ionization. From the chromatogram, the ratio of the area corresponding to 10-undecenoic acid, with respect to the sum of the areas corresponding to the compounds having 11 carbon atoms, is determined. The conversion of 10-undecenoic acid can then be calculated by subtracting this ratio from 1, according to the following formula:

Conversion=1−(area 10−undecenoic acid)/(sum of the areas).

From the chromatogram, the yield is subsequently estimated by determining the ratio of the area corresponding to 11-bromoundecanoic acid, with respect to the sum of the areas corresponding to the compounds having 11 carbon atoms in the aliquot, according to the following formula:

Yield=(area 11−bromoundecanoic acid)/(sum of the areas)

The selectivity can then be estimated according to the formula: Selectivity=Yield/Conversion

TABLE 1
Parameters of the process

| | HBr introduced into | HBr injected into the | Reactor | |
|---|---|---|---|---|
| | the process/10- | reaction mixture/10- | temper- | HBr |
| Exam- | decenoic acid molar | decenoic acid molar | ature | recycl- |
| ple | flow rate ratio | flow rate ratio | [° C.] | ing |
| 1 | 1.15 | 1.85 | 24 | Yes |
| 2 | 1.5 | 1.5 | 24 | No |
| 3 | 1.4 | 1.4 | 24 | No |
| 4 | 1.3 | 1.3 | 24 | No |
| 5 | 1.05 | 1.8 | 24 | Yes |
| 6* | 1.15 | 1.9 | 20 | Yes |
| 7* | 1.85 | 1.85 | 20 | No |

*modified process

The results of analysis of the aliquot and of the performance qualities of the reaction are given in table 2.

The ratio of the molar flow rate of gaseous HBr injected into the reaction mixture (equal to the sum of the molar flow rate of HBr introduced into the process (5) and of the molar flow rate of recycled HBr (6)), with respect to the molar flow rate of 10-undecenoic acid, is estimated at 1.85.

After 65 minutes, an aliquot is withdrawn at the outlet of the separation vessel and subjected to analysis by gas chromatography. The selectivity for 11-bromoundecanoic acid is 95%, and thus identical to that at the outlet of the hydrobromination reactor, and the yield is 94.9%.

TABLE 2
Concentration of residual HBr and yield of the process

| | Concentration | | | |
|---|---|---|---|---|
| | residual HBr | Yield | Selectivity | Conversion |
| Example | [% by weight] | [%] | [%] | [%] |
| 1 | 4.1 | 94.6 | 95 | >99 |
| 2 | 3.1 | 94 | 94.4 | >99 |
| 3 | 2.4 | 92.4 | 93.1 | >99 |
| 4 | 1.8 | 84 | 87 | 97 |
| 5 | N.D. | 94.5 | 94.7 | >99 |
| 6* | N.D. | 94.5 | 95 | >99 |
| 7* | N.D. | 94.3 | 94.8 | >99 |

*modified process

### Example 2

Example 1 is reproduced with the same appliance but in which the second vesssel (9) and the vent recycling line (6) have been removed. The ratio of the flow rate of gaseous HBr injected into the reaction mixture in mol/h to the flow rate of 10-undecenoic acid in mol/h is adjusted to 1.5.

The results are given in table 2.

### Example 3

Example 2 is reproduced but while adjusting the ratio of the flow rate of gaseous HBr injected into the reaction mixture in mol/h to the flow rate of 10-undecenoic acid in mol/h to a value of 1.4.

The results are given in table 2.

### Example 4

Example 2 is reproduced with a ratio of the flow rate of gaseous HBr injected into the reaction mixture in mol/h to the flow rate of 10-undecenoic acid in mol/h adjusted to 1.3.

The results are given in table 2.

### Example 5

Example 1 is reproduced while replacing the HBr injected at (5) with an HBr/hydrogen/HCl mixture in the volume ratio 90/4/1 and while ensuring a ratio of the flow rate of HBr introduced into the process at (5) in mol/h (not counting the hydrogen or the HCl) to the flow rate of 10-undecenoic acid in mol/h of 1.05.

The results are given in table 2.

### Example 6

Example 1 is reproduced with the following changes:
Instead of injecting a solution of 10-undecenoic acid in cyclohexane at (7), a stream of molten 10-undecenoic acid at 50° C. and a stream of methylcyclohexane at ambient temperature are injected, in the proportions by weight 15/85, into the circuit between the discharge of the pump and the liquid inlet of the venturi.
The vessel (11) is maintained at 20° C.
The vessel (9) is replaced by a column comprising a packing and a reboiler at the bottom, regulated at 100° C., in which the liquid stream coming from the vessel is injected at the top, the reaction liquid is withdrawn at the bottom by the pump (10) in order to maintain a constant level in the reboiler, with a volume of liquid of 0.06 liter, and the gaseous vent at the top of the column is returned to the gas suction of the venturi.

The results at the outlet of the reactor are given in table 2.

An analysis by argentometry and by chromatography makes it possible to show that the liquid reaction mixture at the outlet of the pump (12) contains less than 0.1% of HBr and that the yield at the outlet of the pump (12) is 94.8%.

### Example 7

Example 2 is reproduced while replacing the vessel (11) and the venturi and the liquid loop with the pump by a stirred jacketed vessel with a self-priming turbine. The stream of HBr (5) is fed in under the stirring rotor. The ratio of the flow rate of gaseous HBr injected into the reaction mixture in mol/h to the flow rate of 10-undecenoic acid in mol/h is adjusted to 1.85 and the temperature in the vessel (11) is maintained at 20° C.

The results are given in table 2.

The combined results demonstrate that the hydrobromination can be carried out in a solvent different from toluene and benzene, with an injection of HBr in gas form, at ambient temperature and with a short residence time, and can provide a highly satisfactory yield.

It is observed that, the greater the excess of HBr injected into the reaction mixture and the greater the concentration of HBr in the reaction medium exiting from the reactor, the better the selectivity.

It is furthermore observed that the recycling of the HBr makes it possible to achieve a high selectivity with a reduced flow rate of HBr introduced into the process. Recycling can be provided, for example, by means of an item of separation equipment and a vent recycling line. More specifically, it is possible to achieve a selectivity of 95% and while working in a molar excess of HBr of 5 mol % to 15 mol %, whereas, in the absence of recycling, the HBr has to be used in a molar excess of more than 50%.

Furthermore, it has been possible to validate that it is possible, in the context of the process of the invention, to use HBr having a residual content of hydrogen or HCl, without substantially affecting the yield (see example 5).

Moreover, it is found that the removal of the HBr in the reaction mixture by means of a column with packing and reboiler at the bottom makes it possible to markedly reduce the residual content of HBr without degrading the yield of the reaction (see example 6).

Finally, it has been confirmed that the use of other gas-liquid mixers than the venturi, for example of a self-priming turbine, makes it possible to obtain equivalent results.

### LIST OF THE DOCUMENTS CITED

FR 928 265
Semyonov et al., Maslozhirova Promyshlennost, 1971, Vol. 37, pp. 31-33
CN 103804209 B
EP 3 030 543 B1

1. A process for the continuous synthesis of a compound of formula (II) Br—(CH₂)ₙ+₂—COOR, comprising a stage consisting of:
(a) the hydrobromination of a compound of formula (I) CH₂═CH—(CH₂)ₙ—COOR:
where, in the formulae (I) and (II), n is an integer of between 7 and 9, and R is chosen from H or a linear or branched alkyl radical comprising from 1 to 10 carbon atoms, in particular methyl, ethyl, isopropyl or propyl,
with HBr in the presence of a radical initiator and of at least one solvent;
said process being characterized in that the reaction is carried out in the absence of benzene and of toluene and in that, in stage (a), the HBr is injected into the reaction mixture in the gaseous form and in stoichiometric excess.

2. The process as claimed in claim 1, characterized in that the ratio of the molar flow rate of HBr injected in stage (a) to the molar flow rate of the compound of formula (I) injected in stage (a) is from 1.2 to 3, preferably from 1.3 to 2.2, more preferentially from 1.4 to 2 and in particular from 1.5 to 1.9.

3. The process as claimed in claim 1 or 2, characterized in that the outlet stream from the reactor of the liquid reaction mixture on conclusion of stage (a) comprises at least 2%, preferably at least 3%, and more preferentially at least 3.5% and in particular at least 4% by weight of HBr.

4. The process as claimed in one of claims 1 to 3, characterized in that the process additionally comprises the subsequent stages consisting of:
(b) the separation of the excess HBr from the liquid reaction mixture resulting from stage (a);
(b1) optionally, separation of the excess HBr from the gaseous reaction mixture resulting from stage (a); and
(c) the recycling of the HBr separated in stage (b) and (b1) if appropriate to stage (a).
and
(c) the recycling of the HBr separated in stage (b) to stage (a).

5. The process as claimed in one of claims 1 to 4, comprising the stages consisting of:
(a1) introduction of the compound of formula (I), of the HBr, of the initiator and of the solvent(s) into a first reactor at an appropriate temperature for an appropriate residence time;
(a2) withdrawal of the reaction mixture from the first reactor and introduction into an item of separation equipment; if appropriate, followed by a subsequent stage of:
(b) separation of the residual HBr from the reaction mixture; and
(c) recycling of the separated HBr to stage (a1).

6. The process as claimed in one of claims 1 to 5, characterized in that stage (a) is carried out at a temperature of between 5 and 50° C., preferably between 10 and 40° C. and very particularly from 20 to 30° C.

7. The process as claimed in one of claims 1 to 6, characterized in that the radical initiator is molecular oxygen used as is or as a mixture with inert gases, for example air or oxygen-enriched air.

8. The process as claimed in claim 4 to 7, characterized in that the first reactor is a stirred vessel with a self-priming turbine or a jet loop reactor comprising a venturi.

9. The process as claimed in one of claims 4 to 8, characterized in that the item of separation equipment is a stirred vessel or a column.

10. The process as claimed in one of claims 1 to 9, characterized in that it is carried out in the absence of aromatic solvent.

11. The process as claimed in one of claims 1 to 10, characterized in that the product of formula (I) is chosen from 11-bromoundecanoic acid, 10-bromodecanoic acid and 9-bromononanoic acid.

12. The process as claimed in one of claims 1 to 11, characterized in that the solvent is chosen from cyclohexane, methylcyclohexane, heptane, methylcyclopentane, n-hexane, 2-methylhexane, 3-methylhexane, n-heptane, isooctane, petroleum ether, tetralin, 1,1,1-trichloroethane, dibromoethane, chloroform, carbon tetrachloride, tetrachlorethylene, 1-bromopropane, dimethyl carbonate, tetrahydrofuran, 1,4 dioxane, 2-methyltetrahydrofuran, tetrahydropyran, 1-propoxypropane, 1-ethoxybutane, 2-isopropoxypropane, acetonitrile and their mixtures.

13. The process as claimed in one of claims 4 to 12, additionally comprising, during stage (b) of separation of the excess HBr from the gaseous reaction mixture resulting from stage (a).

14. A process for the synthesis of a compound of formula (III) NH₂—(CH₂)ₙ+₂—COOR, comprising a stage consisting of:
(i) ammonolysis on the compound of formula (II) obtained by the process as claimed in one of claims 1 to 13; and
(ii) separation of the compound of formula (III) NH₂—(CH₂)ₙ+₂—COOR formed.

15. A process for the synthesis of a polyamide or copolyamide comprising the stage of polycondensation of the compound of formula (III) obtained by the process as claimed in claim 14, alone or as a mixture with other monomers.
